(19) United States
(12) Patent Application Publication
(10) Pub. No.: US 2005/0137468 A1
(43) Pub. Date: Jun. 23, 2005

(54) Device, system, and method for in-vivo sensing of a substance

(76) Inventors: Jerome Avron, Haifa (IL); Kevin Rubey, Ventura, CA (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 11/014,806

(22) Filed: Dec. 20, 2004

Related U.S. Application Data
(60) Provisional application No. 60/530,246, filed on Dec. 18, 2003.

(51) Int. Cl. A61B 1/00; A61B 5/00

(52) U.S. Cl. 600/310; 600/103

(57) ABSTRACT

An in vivo device and a method for imaging, monitoring and/or sensing of a substance in a body lumen. The in vivo device may include a holding unit, for example a cap for holding the substance, and sensing means, for example a sensor, an imager and an illumination source, for capturing various information in relation to the changes occurring in the substance.

### RELATED APPLICATION DATA

This application claims benefit from U.S. provisional application Ser. No. 60/530,246, filed on Dec. 18, 2003, entitled DEVICE, SYSTEM, AND METHOD FOR IN-VIVO SENSING OF A SUBSTANCE which is incorporated in its entirety by reference.

### FIELD OF THE INVENTION

The present invention relates to the field of in-vivo sensing. More specifically, the present invention relates to devices, systems, and methods for in-vivo sensing, monitoring or imaging of a substance.

### BACKGROUND OF THE INVENTION

Devices, systems and methods for in-vivo sensing of passages or cavities within a body, and for gathering information (e.g., image information, pH information, temperature information, electrical impedance information, pressure information, etc.), are known in the art.

An in-vivo sensing device may include, for example, an imaging device for obtaining images from inside a body cavity or lumen, such as the gastrointestinal (GI) tract. The imaging device may include, for example, an imager associated with units such as, for example, an optical system, an illumination source, a power source, a transmitter and an antenna. Other types of in-vivo sensing devices exist, such as endoscopes which may not require a transmitter, and devices performing functions other than imaging.

A substance (e.g., a drug, a food, a chemical substance, etc.) may be inserted into a patient's body, and may dissolve or otherwise change one or more of its properties while in the GI tract. Typically, known in vivo sensing devices can not monitor a change in a substance while in the GI tract or in other body lumens.

There is therefore a need for a device, system and method for capturing information of a substance within lumens or cavities or in other locations within the body.

### SUMMARY OF THE INVENTION

There is provided, in accordance with some embodiments of the present invention, a device, system, and method for sensing, monitoring and/or imaging of a substance in-vivo and/or in-vitro. According to one embodiment of the invention there may be provided, in an in vivo device one or more sensors, a holding unit or compartment such as formed by a basket or a cap which may hold a substance, for example a drug, a food or a chemical matter which may dissolve when placed in a digestive system of a human body or in a specific part of the digestive system, e.g., in the stomach, in the large intestine or in other parts.

According to one embodiment the in vivo device may sense, image, monitor and/or otherwise capture various information or properties in relation to the dissolving of the substance, for example images of the substance as it dissolves, temperature, pH level etc. The information may be analyzed by a data processor which may produce conclusions related to the dissolving or changing of the substance.

Embodiments of the invention may allow various other benefits, and may be used in conjunction with various other applications.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a schematic illustration of an in-vivo sensing system in accordance with an embodiment of the invention;

FIG. 2 is a schematic illustration of an in-vivo sensing device in accordance with an embodiment of the invention; and

FIG. 3 is a flow-chart diagram of a method of in-vivo sensing in accordance with an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well known features may be omitted or simplified in order not to obscure the invention.

It should be noted that although a portion of the discussion may relate to in-vivo imaging devices, systems, and methods, the present invention is not limited in this regard, and embodiments of the present invention may be used in conjunction with various other in-vivo sensing devices, systems, and methods. For example, some embodiments of the invention may be used, for example, in conjunction with in-vivo sensing of pH, in-vivo sensing of temperature, in-vivo sensing of pressure, in-vivo sensing of electrical impedance, in-vivo detection of a substance or a material, in-vivo detection of a medical condition or a pathology, in-vivo acquisition or analysis of data, and/or various other in-vivo sensing devices, systems, and methods.

Some embodiments of the present invention are directed to a typically swallowable in-vivo sensing device. Devices according to embodiments of the present invention may be similar to embodiments described in International Application WO 01/65995 entitled A DEVICE AND SYSTEM FOR IN-VIVO IMAGING and/or in U.S. Pat. No. 5,604,531 entitled IN-VIVO VIDEO CAMERA SYSTEM, each of which are assigned to the common assignee of the present invention and each of which are hereby incorporated by reference. Furthermore, a receiving and/or display system which may be suitable for use with embodiments of the present invention may also be similar to embodiments described in WO 01/65995 and/or in U.S. Pat. No. 5,604,531. Of course, devices and systems as described herein may have other configurations and/or other sets of components. For example, the present invention may be practiced using an endoscope, needle, stent, catheter etc.

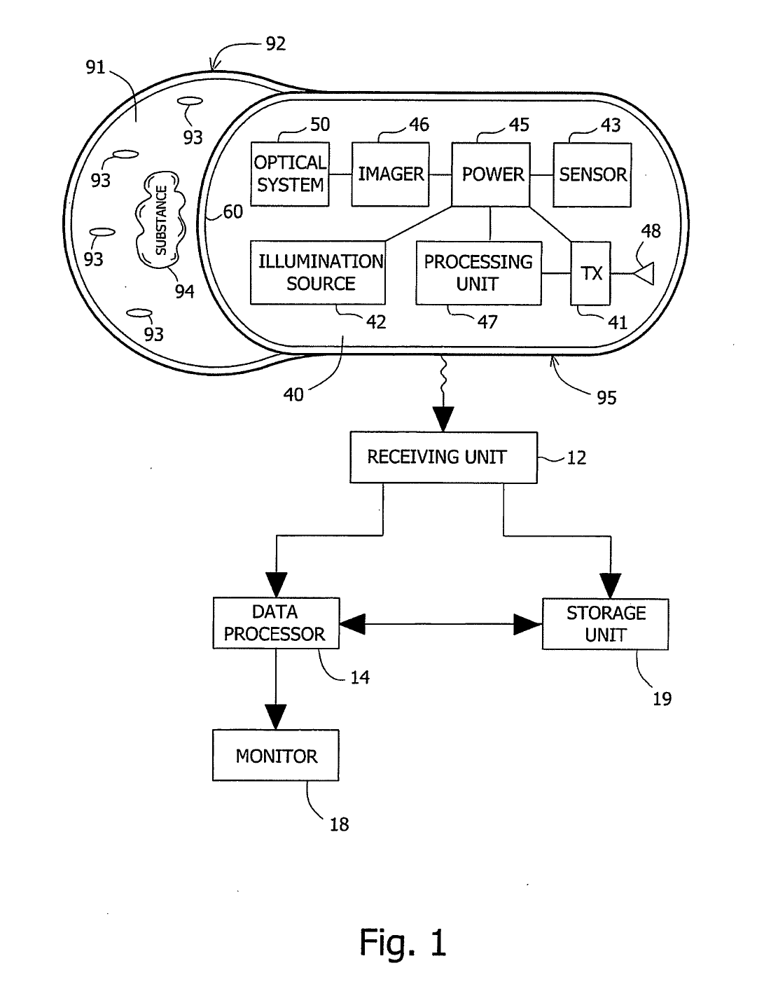

FIG. 1 shows a schematic diagram of an in-vivo and/or in-vitro sensing system in accordance with an embodiment of the present invention. In one embodiment, the system may include a device 40 having an imager 46, one or more illumination source(s) 42, and a transmitter 41. In some embodiments, device 40 may be implemented using a swallowable capsule, but other sorts of devices or suitable implementations may be used. Device 40 may communicate with an external receiving and display system to provide display of data, control, or other functions. For example, power may be provided by an internal battery or a wireless receiving system. Other embodiments may have other configurations and capabilities. For example, outside a patient's body may be a receiving unit, for example, a receiving unit 12 (including, for example, an antenna or an antenna array), a storage unit 19, a data processor 14, and an output device, such as a monitor 18 or other suitable data displaying apparatus, which may display output data, such as image data or other data. Device 40 may include a container or holding unit, such as basket or cap 91, holding a possibly dissolvable substance 94. A transparent or partially transparent portion 60 may separate components of device 40 such as imager 46, which may be sealed within shell 95, and cap 91, which may be open to the outside environment e.g., letting fluids in. Transparent portion 60 may be integral with shell 95, or may be a separate piece.

Monitor 18 may include, for example, one or more screens, monitors, or suitable display units. Monitor 18, for example, may display images captured and/or transmitted by device 40, control data, position data (e.g., data describing or indicating the location or the relative location of device 40), and various other suitable data. In some embodiments, for example, monitor 18 may display images of a substance, a dissolving substance or a changing substance (e.g., substance 94 described herein), or data related to such substances. In one embodiment, for example, both an image and its position or location may be presented using a single monitor, or using a plurality of monitors. Other systems and methods of storing and/or displaying collected image data may be used. In addition, components may be distributed over multiple sites or units.

Transmitter 41 may be a wireless transmitter, e.g., operate wirelessly by using radio waves, IR or other known wireless transmission technologies; but in some embodiments, such as those where device 40 is or is included within an endoscope, transmitter 41 may transmit via, for example, wire optical fiber and/or other suitable methods.

Device 40 typically may be or may include an autonomous swallowable capsule, but device 40 may have other shapes and need not be swallowable or autonomous. Embodiments of device 40 are typically autonomous, and are typically self-contained. For example, device 40 may be a capsule or other unit where all the components are substantially contained within a container or shell, and where device 40 does not require any wires or cables to, for example, receive power or transmit information.

In one embodiment, device 40 may include an imager 46, for example an in-vivo video camera, which may capture and transmit images of, for example, the GI tract while device 40 passes through the GI lumen. In other embodiments the imager need not capture images of the body, but may rather more or less exclusively image the substance 94. Other lumens and/or body cavities may be imaged and/or sensed. In some embodiments, imager 46 may include, for example, a Charge Coupled Device (CCD) camera or imager, a Complementary Metal Oxide Semiconductor (CMOS) camera or imager, a digital camera, a still camera, or other suitable imagers, cameras, or image acquisition components.

In one embodiment, imager 46 in device 40 may be operationally connected to transmitter 41. Transmitter 41 may transmit images to, for example, receiving unit 12, which may send the data to data processor 14 and/or to storage unit 19. Transmitter 41 may also include control capability, although control capability may be included in a separate component. Transmitter 41 may include any suitable transmitter able to transmit image data, other sensed data, and/or other data (e.g., control data) to a receiving device. For example, transmitter 41 may include an ultra low power Radio Frequency (RF) high bandwidth transmitter, possibly provided in Chip Scale Package (CSP). Transmitter 41 may transmit via antenna 48. Optionally, transmitter 41 and/or device 40 may include a controller, which may include one or more control modules, circuitry and/or functionality for controlling device 40 and/or for controlling the operational mode or settings of device 40.

Power source 45 may include one or more batteries. For example, power source 45 may include silver oxide batteries, lithium batteries, other suitable electrochemical cells having a high energy density, or the like. Other power sources may be used. For example, power source 45 may receive power or energy from an external power source (e.g., a power transmitter), which may be used to transmit power or energy to device 40.

Data processor 14 may analyze the data received from device 40, and may be in communication with storage unit 19, transferring frame data to and from storage unit 19 Data processor 14 may also provide the analyzed data to monitor 18, where a user (e.g., a physician, and/or a scientist) may view the data. Monitor 18 may display one or more images of the GI lumen (or of other imaged body lumen or cavity), and/or the position or location in the GI tract (or other body lumen or cavity) at which the image or images were taken. In some embodiments, for example, monitor 18 may display images of a substance, a dissolving substance or a changing substance (e.g., substance 94 described herein), or data related to such substances. In one embodiment, data processor 14 may be configured for real time processing and/or for post processing to be performed and/or viewed at a later time. In the case that control capability (e.g., delay, timing, etc) is external to device 40, a suitable external device (such as, for example, data processor 14 or receiving unit 12) may transmit one or more control signals to device 40.

In some embodiments, in addition to or instead of revealing pathological or other conditions of the GI tract, the system may provide information about the location of these conditions, or about the location of a substance (e.g., a dissolving or changing substance). Suitable tracking devices and methods are described in embodiments of the above-mentioned U.S. Pat. No. 5,604,531 and/or U.S. Patent Application Publication No. U.S.-2002-0173718-A1, filed on May 20, 2002, titled “Array System and Method for Locating an In-Vivo Signal Source”, assigned to the assignee of the present invention, and fully incorporated herein by reference. Other suitable location identification systems and methods may be used in accordance with embodiments of the present invention.

Optionally, device 40 may include a processing unit 47, for example, to process signals and/or data generated by imager 46. In some embodiments, processing unit 47 need not be a separate component; for example, processing unit 47 may be integral to imager 46, to transmitter 41, to another component, or may not be needed. In some embodiments processing may be done in a component that is external to the body.

In some embodiments, device 40 may include one or more illumination sources 42, for example one or more white LEDs or any other suitable light sources. Illumination sources 42 may, for example, illuminate a body lumen being imaged and/or sensed. In some embodiments, for example, illumination sources 42 may, for example, illuminate a substance, a dissolving substance or a changing substance (e.g., substance 94 described herein). An optical system 50, including, for example, one or more optical elements, such as one or more lenses or composite lens assemblies, one or more suitable optical filters, or any other suitable optical elements, may aid in focusing reflected light onto imager 46 and/or performing other light processing operations.

Typically, device 40 may transmit image information in discrete portions. Each portion may typically correspond to an image or a frame; other suitable transmission methods may be used. For example, in some embodiments, device 40 may capture and/or acquire an image once every half second, and may transmit the image data to receiver 12. Other constant and/or variable capture rates and/or transmission rates may be used.

Typically, the image data recorded and transmitted may include digital color image data; in alternate embodiments, other image formats (e.g., black and white image data) may be used. In one embodiment, each frame of image data may include 256 rows, each row may include 256 pixels, and each pixel may include data for color and brightness according to known methods. For example, in each pixel, color may be represented by a mosaic of four sub-pixels, each sub-pixel corresponding to primaries such as red, green, or blue (where one primary is represented twice). The brightness of the overall pixel may be recorded by, for example, a one byte (e.g., 0-255) brightness value. Other suitable data formats may be used, and other suitable numbers or types of rows, pixels, sub-pixels and/or colors may be used.

Optionally, device 40 may include one or more sensors 43, instead of or in addition to a sensor such as imager 46. Sensor 43 may, for example, sense, detect, determine and/or measure one or more values of properties or characteristics of the surrounding of device 40 and/or of substance 94. For example, sensor 43 may include a pH sensor, a temperature sensor, an electrical conductivity or impedance sensor, a pressure sensor, or any other known suitable in-vivo sensor. In some embodiments, sensor 43 may be placed, situated and/or positioned substantially adjacent to the outer wall or shell of device 40, for example, so as to maximize or increase the exposure of sensor 43 to the in-vivo conditions outside of such wall of device 40.

In some embodiments, components within device 40 may be connected and/or operatively connected, for example, using a wired or wireless connection. In one embodiment, optionally, some components within device 40 may be operatively connected to, or included or embedded within, an Application Specific Integrated Circuit (ASIC). In alternate embodiments, components within device 40 (e.g., imager 46, or sensor 43) may be operatively linked and/or connected to each other without an ASIC, for example, using a wired connection, a wireless connection, a microwave connection, or other suitable connections.

Device 40 may include a holding unit, basket or cap 91. In one embodiment, holding unit, basket or cap 91 may include or may be, for example, a compartment, a head, a basket, a pod, a nodule, a bubble, a cover, a case, a casing, or another suitable container. Cap 91 may be external to device 40, for example, cap 91 may be attached or otherwise bonded to a side of device 40. In one embodiment, cap 91 may be located and/or positioned to be in proximity, or in relative proximity, to imager 46, to optical system 50, to illumination source 42, or to a region of device 40 which includes one or more of these components. The imager 46 may be configured or aligned so that it may image at least part of the compartment or space formed by the cap 91. Illumination source(s) 42 may be aligned so that at least a portion of the illumination given off is reflected by substance 94. Typically, illumination source(s) 42 illuminate substance 94 via transparent portion 60, and imager 46 may view substance 94 via transparent portion 60. In some embodiments, cap 91 may be detachable from device 40, for example so that substances may be inserted or taken out. For example, cap 91 may be attached to and detached from device 40, e.g., using a pressure mechanism, a bonding mechanism, a holding mechanism, a screw thread mechanisms, one or more screws or holders, glue, soldering or other suitable mechanisms. In alternate embodiments, for example, cap 91 may be integrated with a shell 95 of device 40, or cap 91 may be an integral and/or integrated part of shell 95 of device 40. A door or latch may be provided on cap 91, so that substances may be inserted or taken out.

In some embodiments, cap 91 may include a shell 92. Shell 92 may include or may be, for example, a transparent, semi-transparent or non-transparent layer or cover; cap 91 may be transparent, semi-transparent or non-transparent. In some embodiments, shell 92 may include one or more holes or pores 93. Holes or pores 93 may include various suitable outlets, gaps, slits, openings and/or punctures, which may have various suitable sizes and shapes. Although FIG. 1 shows four circular holes 93, other number of holes 93 may be used, and various other shapes of holes 93 (e.g., square, rectangular, oval, triangular, etc.) may be used; similarly, the location or relative positioning of holes 93 in shell 92 may be different from the example shown in FIG. 1.

In some embodiments, cap 91 and shell 92 may be formed of, for example, plastic, transparent or semi-transparent plastic, glass, plastic-glass, isoplast™, etc. In one embodiment, for example, cap 91 and/or shell 92 need not be transparent, and device 40 may sense and/or image a substance contained within cap 91, shell 92 and/or device 40.

In some embodiments, cap 91 may include or contain a substance 94. Substance 94 may include, for example, a drug, a compound, a pharmaceutical drug or compound, a chemical substance, a test substance or drug, an experimental substance or drug, a food, a medicine, a medication, a dye, a pill, a tablet, a softgel, a sphere, a capsule, or a plurality of such materials. Substance 94 may include, for example, solid matter, liquid matter, blended or mixed matters, with various shapes, sizes, weights, colors or properties.

In some embodiments, substance 94 may dissolve, solidify, liquefy, turn into liquid or solid, soften, harden, change color, blend, mix, or otherwise change one or more of its properties when inserted into a body of a human being or other living being, or when inserted into a body organ, lumen or region of such a body of a human being or other living being. For example, in some embodiments, substance 94 may include a drug, a food or a chemical matter which may dissolve when placed in a digestive system of a human body or in a specific part of the digestive system, e.g., in the stomach, in the large intestine or in other parts.

In one embodiment, holes 93 may be sufficiently large to allow one or more body liquids (e.g. blood, digestive system fluids, etc.) to touch substance 94 and to affect, such to dissolve or further dissolve substance 94. Holes 93 may be sufficiently large to allow such body liquids to enter into cap 91 and/or to exit cap 91. In some embodiments, holes 93 may have properties (e.g., size, number, position, shape, etc.) to allow a graduate, slow or rapid dissolution of substance 93 into the body environment which surrounds device 40 or cap 91. Typically, when initially inserted into cap 91, substance 94 may be a block, pill or mass which cannot pass through holes 93; this may change as dissolving or breaking apart occurs.

In some embodiments, substance 94 may dissolve, or gradually dissolve, into the body environment in which device 40 is located. Device 40 may sense, image, monitor and/or otherwise capture various information or properties in relation to the dissolving of substance 94. For example, in some embodiments, device 40 may image (e.g., using imager 46) and may capture images of substance 94 as it dissolves or otherwise changes its properties. In one embodiment, device 40 may monitor or capture other suitable information in relation to the dissolving of substance 94, for example, temperature, pH level, pressure, position, dissolving speed, dissolving time, dissolving rate, dissolving pattern, properties of substance 94 as it dissolves (e.g., size, dimensions, shape, color, volume, etc.), or the like.

In one embodiment, imager 46 may image substance 94 as it dissolves and/or changes. In an alternate embodiment, imager 46 may image a body organ in which device 40 is located as substance 94 dissolves and/or changes. In another embodiment, imager 46 may image both substance 94 as it dissolves and/or changes, and the body organ in which device 40 is located; this may be performed, for example, using two or more imagers similar to imager 46, or using an imager 46 and/or optical system 50, which may be pointed, positioned and/or focused to allow capturing images of both substance 94 and the body organ in which device 40 is located.

In some embodiments, device 40 may capture images, data and/or information, which may indicate, or which may allow further processing or calculations to indicate, for example, various conclusions, results or data regarding the dissolving or changing of substance 94. Such results may include, for example, that substance 94 dissolves or changes relatively slowly or relatively rapidly in a certain body organ (e.g., the small intestine, the large intestine, etc.); that substance 94 dissolves or changes faster or slower if substance 94 has or does not have certain properties (e.g., shape, size, weight, volume, etc.); that substance 94 dissolves well or does not dissolve well; or other suitable results.

It is noted that in some embodiments, device 40 may include other suitable components. For example, transparent portion 60 may be an optical dome. Device 40 may include other suitable types of domes, optical domes, or components.

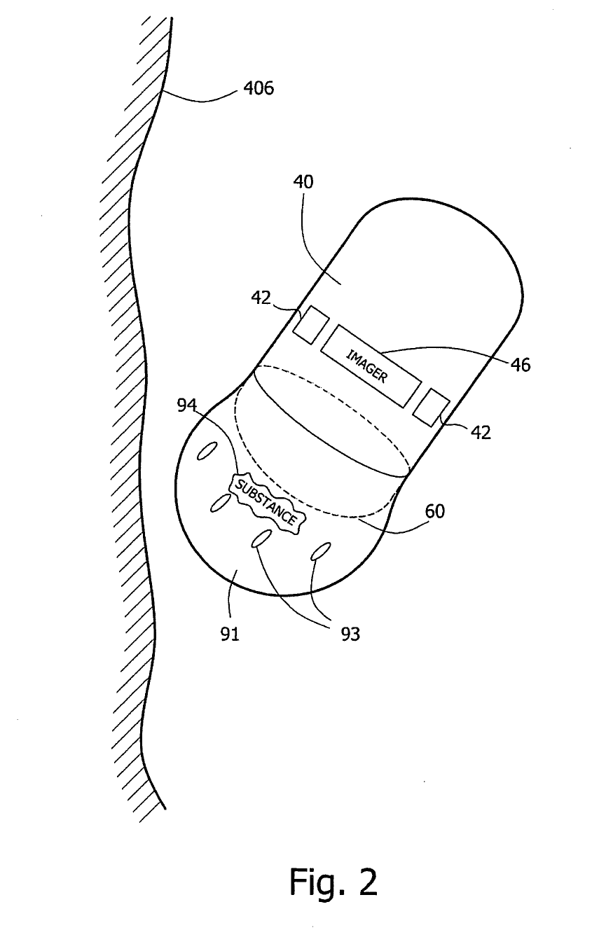

Reference is now made to FIG. 2, which is a schematic illustration of an in-vivo sensing device in accordance with an embodiment of the invention. As shown in FIG. 2, device 40 may include, or may be attached to, cap 91. Cap 91 may include holes or pores 93. It is noted that device 40, holes or pores 93, and/or cap 91 may have various other suitable shapes, dimensions, sizes or proportions.

According to one embodiment, transparent portion 60 may include an optical dome. In one embodiment, the optical dome may typically be transparent; in an alternate embodiment, the optical dome may be semi-transparent. In some embodiments, the optical dome or the transparent portion 60 may be formed of, for example, plastic, transparent or semi-transparent plastic, glass, plastic-glass, isoplast™, etc. In some embodiments, optical dome or the transparent portion 60 may be used as a barrier between the components of device 40 and the contents of cap 91 (e.g., substance 94).

Typically, the contents of cap 91 (e.g., substance 94) may be illuminated (e.g., by illumination sources 42) through the or the transparent portion 60 and an image of the contents of cap 91 may be formed on an imager positioned behind the or the transparent portion 60 (e.g., imager 46).

According to one embodiment, an image may include in-vivo data and data relating to the contents of cap 91. For example, cap 91 may be transparent such that illumination passing through or the transparent portion 60 may also pass through cap 91 illuminating both the contents of cap 91 and, for example, a body lumen wall 406. Thus, an image formed may include data relating to the body lumen and/or to the contents of cap 91 in the body lumen.

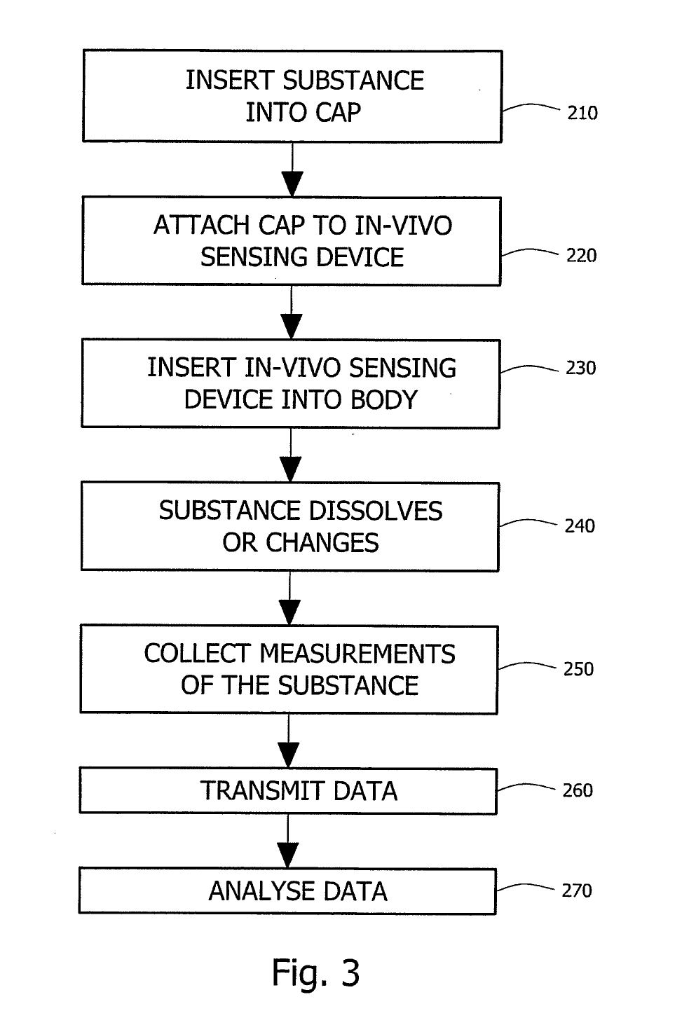

FIG. 3 is a flow-chart diagram of a method of in-vivo and/or in-vitro sensing in accordance with an embodiment of the present invention. The method of FIG. 3, as well as other suitable methods in accordance with embodiments of the invention, may be used, for example, in association with the system of FIG. 1, with one or more in-vivo sensing devices (which may be, but need not be, similar to device 40), and/or with other suitable devices and systems for in-vivo sensing.

In some embodiments, as indicated at block 210, a substance (e.g., substance 94) may be inserted into a cap (e.g., cap 91). Optionally, as indicated at block 220, if the cap is not already attached to an in-vivo sensing device (e.g., device 40), then the cap may be attached and/or bonded to the device. These operations may result in, for example, an in-vivo sensing device 40 which may include cap 91 which may contain substance 94.

As indicated at block 230, the in-vivo sensing device may be inserted into a body of a human being, e.g., into a patient's body. In one embodiment, for example, the device may be implemented using a swallowable capsule, and thus the device (e.g., device 40) may be swallowed.

As indicated at block 240, the substance may dissolve, or may change one or more of its properties. As indicated at block 250, the device may collect measurements of the substance by using, for example a sensor. The collecting process may include for example sensing, imaging and/or monitoring one or more properties of the substance, for example as it dissolves or changes, and/or one or more properties of a body organ surrounding the device as the substance dissolves or changes.

As indicated at block 260, the device may transmit captured data, for example, to receiver 12. As indicated at block 270, the data may be analyzed by a suitable processing unit, for example, by data processor 14. Such analysis may, for example, produce one or more results or conclusions related to the dissolving or changing of the substance.

Some of the operations described above may be optional, and some embodiments may use another suitable order or sequence of operations. Furthermore, other suitable operations or sets of operations may be used in accordance with embodiments of the invention.

It is noted that a device, system and method in accordance with some embodiments of the invention may be used, for example, in conjunction with a substance (e.g., substance 94) which may be inserted into a human body. However, the scope of the present invention is not limited in this regard. For example, some embodiments of the invention may be used in conjunction with a substance which may be inserted into a non-human body, e.g., a dog, a cat, a rat, a cow, or other animals, pets, laboratory animals, farm animals or pet animals. This may allow, for example, monitoring, sensing and/or imaging a substance as it dissolves inside a non-human body or an animal body.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

1. An in-vivo device comprising:
a compartment including a set of openings; and
an imager, the imager configured to image at least part of the compartment.

2. The device according to claim 1, wherein said in-vivo device is an autonomous capsule.

3. The device according to claim 1, comprising a sensor.

4. The device according to claim 3, wherein said sensor is selected from the group consisting of: a pH sensor, a temperature sensor, an electrical conductivity sensor, a pressure sensor.

5. The device according to claim 1, comprising an illumination source.

6. The device according to claim 1, comprising a transmitter.

7. The device according to claim 1, wherein the compartment is configured for containing a substance.

8. The device according to claim 1, wherein the compartment is configured to allow passage of fluids through the openings.

9. The device according to claim 1, wherein the compartment is at least partially transparent.

10. A device for in vivo sensing of a substance, said device comprising:
a holding unit, said holding unit configured for containing at least a substance; and
a transmitter.

11. The device according to claim 10, wherein said holding unit is disposed within said device.

12. The device according to claim 10, wherein said holding unit is detachable from said device.

13. The device according to claim 10, wherein said holding unit is external to said device.

14. The device according to claim 10, wherein said holding unit comprises a shell including at least one opening.

15. The device according to claim 10, wherein said holding unit is selected from the group consisting of: a basket, a cap, a pod, a bubble, or a case.

16. The device according to claim 10, comprising means to allow body liquids to pass in or out of said holding unit.

17. A system for in vivo sensing of a substance, the system comprising:
an autonomous in-vivo sensing device, said sensing device comprising a holding unit, said holding unit configured to contain the substance; and a transmitter; and
an external receiving unit.

18. The system according to claim 17, comprising a data processor.

19. The system according to claim 17, comprising a monitor.

20. A method for in vivo sensing of a substance the method comprising the steps of:
inserting said substance into an in vivo sensing device;
introducing said in vivo sensing device into a patient's body; and
sensing said substance.

21. The method according to claim 20, comprising inserting said substance into a holding unit.

22. The method according to claim 20, comprising imaging said substance.

23. The method according to claim 20, comprising transmitting sensed information to a receiving unit.

24. The method according to claim 20, comprising analyzing said sensed information.
